(19) United States
(12) Patent Application Publication
(10) Pub. No.: US 2018/0166981 A1
(43) Pub. Date: Jun. 14, 2018

(54) POWER CONTROL AND MONITORING ARRANGEMENT

(71) Applicant: KONINKLIJKE PHILIPS N.V., EINDHOVEN (NL)

(72) Inventor: MICHAEL JAMES ROBBERT LEPPARD, HUNSTON (GB)

(21) Appl. No.: 15/580,906

(22) PCT Filed: Jun. 7, 2016
(86) PCT No.: PCT/EP2016/062888

§ 371 Date: Dec. 8, 2017

(30) Foreign Application Priority Data
Jun. 17, 2015 (EP) 15172476.2

(51) Int. Cl. H02M 3/04; H02N 2/18; B08B 3/12; A61M 11/00; G01R 21/00; G01R 19/00; H04R 17/00; H04R 29/00

(52) U.S. Cl. CPC H02M 3/04; H02N 2/181; B08B 3/12; A61M 11/005; G01R 21/00; A61M 2205/50; H04R 17/00; H04R 29/001; A61M 2205/3317; A61M 2205/8262; G01R 19/003

(57) ABSTRACT

Current flow monitoring, from which a power dissipation metric in a load circuit may be obtained, is often performed using a current sense resistor. However, this implies extra circuit complexity. This application discusses a way to derive an indication of power consumption in a situation where an efficient load, is present, without the use of a current sense resistor, or the associated electronics. A nebulizer is an example of a device used for providing a medicament to a patient via their respiratory tract. A nebulizer typically comprises a reservoir for storing the medicament, in fluid communication with a means for converting the medicament to an aerosol. Knowledge of the power dissipation of such devices is useful.

### FIELD OF THE INVENTION

The present invention relates to a voltage converter control and power dissipation monitoring arrangement for monitoring an efficient load, in particular a load comprising a piezoelectric nebulizer circuit. The present invention also relates to, a method for voltage converter control and power dissipation monitoring of an efficient load, a nebulizer system, a piezo-electric speaker system, an ultrasonic cleaning system, a computer program element, and a computer-readable medium.

### BACKGROUND OF THE INVENTION

A power efficient load dissipates most of its power in the form of useful work, rather than resistive losses. One such example of a power efficient load is a piezo-electric element of a nebulizer. A nebulizer is a device used for providing a medicament to a patient via their respiratory tract. A nebulizer typically comprises a reservoir for storing the medicament. The reservoir is in fluid communication with a means for converting the medicament into an aerosol. The conversion means is also in fluid communication with a breathing tube. In use, a patient inhales air through the breathing tube. The medicament aerosol produced by the conversion means is entrained into the stream of air flowing through the breathing tube. This allows an extensive distribution of the medicament throughout the patient's respiratory system. The effectiveness of the process of converting the medicament to an aerosol depends, partly, on the performance of the conversion means.

WO 2015/010809 discusses a nebulizer system. Such systems can be further improved.

### SUMMARY OF THE INVENTION

It would be advantageous to have an improved technique for monitoring the status of a power efficient load, in particular, a nebulizer.

Towards this end, a first aspect of the invention provides a voltage converter control and power dissipation monitoring arrangement for monitoring an efficient load comprising a voltage converter and a processor.

The processor is configured to obtain, via a first input of the processor, an input voltage level of a voltage applied to an input of the voltage converter. The processor is configured to generate a PWM voltage converter control signal for controlling the level of an output voltage of the voltage converter, wherein the output of the voltage converter is configured to provide an output voltage, being a multiple of the input voltage level, to an external load terminal of the voltage converter, based on a voltage level defined by a PWM voltage converter control signal. The PWM voltage converter control signal is input into a voltage control input of the voltage converter, and the voltage converter control and power dissipation monitoring arrangement is configured to generate an averaged voltage signal of the PWM voltage converter control signal.

The processor is configured to generate a load dissipation metric indicative of power dissipated in load circuitry connected to the external load terminal, by multiplying the input voltage level with the averaged voltage signal.

The voltage converter control and power dissipation monitoring arrangement according to this first aspect of the invention enables the provision of a simpler indication of the load power dissipated in a load element connected to an external load terminal of the voltage converter control and power dissipation monitoring arrangement. This is because an indication of the power in the load may be estimated using the PWM voltage converter control signal used to control the voltage converter, and by measuring the supply voltage at the input of the voltage converter control and power dissipation monitoring arrangement. In this way, power dissipated in an external circuit connected to the external load terminal of the voltage converter control and power dissipation monitoring arrangement can be measured indirectly.

According to the first aspect of the invention, a voltage converter control and power dissipation monitoring arrangement can be provided which does not require a plethora of additional external components, such as a current-sense resistor, and its associated instrumentation amplifier and analogue to digital converter (ADC). Instead, components which are already present in a typical step-up power supply may be used. Thus, the power dissipated by a load can be monitored more easily. Many applications require load dissipation feedback for adjusting control parameters.

According to a second aspect of the invention, a nebulizer system is provided. The nebulizer system comprises a nebulizer element, a nebulizer controller, and a voltage converter control and power dissipation monitoring arrangement as previously described. A power supply of the nebulizer element is connected to the external load terminal of the voltage converter comprised in the voltage converter control and power dissipation monitoring arrangement. The load dissipation metric calculated by the voltage converter control and power dissipation monitoring arrangement is used by the nebulizer controller to control a nebulization signal applied to the piezo-electric nebulizer element.

Nebulizers are usually hand-held devices, and miniaturization is often preferred, along with a reduction in component complexity. In a nebulizer, the nebulizing element, for instance a mesh element provided with micro apertures, is usually driven by a high-efficiency transducer, such as a piezo-electric element, and these are very efficient. In the process of converting the liquid into an aerosol, very little power is wastefully dissipated as heat or sound. Thus, it can be assumed that substantially all of the power dissipated by a high-efficiency transducer (load) is linked to the nebulization process. The load monitoring circuit discussed above may, therefore, accurately monitor the power dissipated in a nebulizer without the use of a current sense resistor. A nebulizer system can benefit in a reduction of complexity when using a power-monitoring approach as outlined above.

When the voltage converter control and power dissipation monitoring arrangement for monitoring an efficient load is applied to a nebulizer circuit, the load dissipation metric can be used to control the nebulizer's “dry detection mechanism”. In other words, a load element of the nebulizer has a certain impedance, which changes when drugs are being nebulized, and when the drug has run out. The impedance of a nebulizer element, for example, is related to power dissipation. A change in a load dissipation metric could indicate that a nebulizer drug reservoir is dry. The load dissipation metric can also be used to tune the performance of a nebulizer's operating frequency. A control means can “sweep” the nebulizer frequency, and use the maximum or minimum of the load dissipation metric to identify a desired nebulizer performance. Then, the nebulizer is driven at this frequency.

According to a third aspect of the invention, there is provided an ultrasonic cleaning system, comprising an ultrasonic cleaning element housed in an ultrasonic cleaning bath, an ultrasonic cleaning controller, and a voltage converter control and power dissipation monitoring arrangement for monitoring an efficient load as previously described.

The external load terminal of the voltage converter comprised in the voltage converter control and power dissipation monitoring arrangement for monitoring an efficient load is connected to an ultrasonic cleaning element, and the load dissipation metric calculated by the voltage converter control and power dissipation monitoring arrangement for monitoring an efficient load is used by the ultrasonic cleaning controller to control the amplitude of a cleaning signal applied to the ultrasonic cleaning element.

Ultrasonic cleaning systems use high-efficiency transducers, such as piezo-electric elements, to provide the ultrasonic cleaning signal applied to items for cleaning which are placed in an ultrasonic cleaning bath. As previously described, a high-efficiency transducer such as a piezo-electric element loses most power in the form of useful work, rather than as thermal, or other parasitic losses. Therefore, an ultrasonic cleaning system employing a voltage converter control and power dissipation monitoring arrangement as discussed above can be simplified in complexity.

According to a fourth aspect of the invention, there is provided a piezo-electric speaker system. The system comprises: a piezo-electric loudspeaker element, a loudspeaker controller, and a voltage converter control and power dissipation monitoring arrangement for monitoring an efficient load as previously disclosed.

The external load terminal of the voltage converter comprised in the voltage converter control and power dissipation monitoring arrangement for monitoring an efficient load is connected to the piezo-electric loudspeaker element, and the load dissipation metric calculated by the voltage converter control and power dissipation monitoring arrangement for monitoring an efficient load is used by the loudspeaker controller to control the amplitude of an audio signal applied to the piezo-electric loudspeaker element.

A piezo-electric loudspeaker element does not experience substantial parasitic losses. Therefore, the voltage converter control and power dissipation monitoring arrangement is applicable to a piezo-electric speaker system, and allows the power dissipated through sound broadcasting to be monitored in a simple way.

According to a fifth aspect of the invention, there is provided a voltage converter control and power dissipation monitoring method for monitoring an efficient load, comprising the steps of:

a) obtaining an input voltage level of a voltage applied to an input of a voltage converter;
b) generating a PWM voltage converter control signal for controlling the level of an output voltage;
c) providing an output voltage, being a multiple of the input voltage level, to an external load terminal based on a voltage level defined by the PWM voltage converter control signal;
d) generating an averaged voltage of the PWM voltage converter control signal;
e) generating a load dissipation metric indicative of power dissipated in an external load connected to the external load terminal by multiplying the input voltage level with the averaged voltage.

Thus, a load power monitoring method is provided which enables the simpler monitoring of power dissipation.

According to a sixth aspect of the invention, there is provided a computer program element for controlling a voltage converter control and power dissipation monitoring arrangement according to the previous description, which, when being executed by a processing unit, is adapted to perform the method steps discussed above.

According to a seventh aspect of the invention, there is provided a computer-readable medium having stored the above computer program element.

In the following specification, the term “load dissipation metric” means a numerical value representing the power dissipated in a load connected to a voltage converter control and power dissipation monitoring arrangement. The power is dissipated substantially due to useful work being performed by electrical power output by a voltage converter.

In the following specification, the term “voltage converter” means a circuit which enables the conversion of an input voltage to a different output voltage. The voltage converter may be a “step-up” or “boost” converter. Alternatively, the voltage converter may be a step-down converter.

In the following specification, the term “signal smoother” refers to a circuit arrangement which provides an average value of an input signal. Thus this may also refer to a “signal averager”. Thus, a square wave input of 50% mark-space ratio input into a signal smoother (averager) according to this definition would result in a DC output level of 50% of a maximum square wave voltage level. The signal smoother may be implemented using analogue electronic components, such as a RC network. Alternatively, the same functionality could be provided by inputting the signal input to a processor, and calculating the average value using digital arithmetic, implemented on a processor. Thus, the signal smoother generates a smoothed voltage signal, in other words, an average voltage of a signal input into it.

In the following description, the term “efficient load” is taken to mean a load in which most of the power is dissipated in the form of useful work (such as energy used in converting a nebulized medicament to an aerosol), rather than wasted energy. Specifically, an efficient load may be considered to be a load capable of operating at above 80 percent efficiency, more preferably above 90 percent efficiency, and more preferably still, above 95 percent efficiency.

Thus, it is seen as a consideration of the invention to exploit the relative power efficiency of certain load elements in circuits. The use of such efficient load elements allows substantially all of the electrical power delivered to such efficient elements to be converted into useful work. In such a case, there will be very little parasitic dissipation from the load, and it is possible to rely on an assumption that substantially all of the power dissipated in the load element is applied as useful work in an application, for example in a nebulizer, in an ultrasonic cleaning bath, or in a piezo-electric speaker system. This allows the useful calculation of a load dissipation metric. Therefore, such systems can be usefully controlled with a simpler control circuit.

These, and other aspects of the invention, will become apparent from, and are elucidated, with reference to the embodiments described hereinafter.

### BRIEF DESCRIPTION OF THE DRAWINGS

FIG. 1 shows a voltage converter control and power dissipation monitoring arrangement according to an aspect of the invention.

FIG. 2 shows a voltage converter control and power dissipation monitoring arrangement according to an example.

FIG. 3 shows an alternative voltage converter control and power dissipation monitoring arrangement according to an example.

FIG. 4 shows smoothed voltage signals.

FIG. 5 shows a nebulizer system.

FIG. 6 shows an ultrasonic cleaning system.

FIG. 7 shows a method for load power monitoring according to an aspect of the invention.

### DETAILED DESCRIPTION OF EMBODIMENTS

Conventionally, the power dissipated in a load is measured using a current sense resistor. The current sense resistor is placed in series with the load. When a current flows through the current sense resistor, a first voltage drop appears across the terminals of the current sense resistor. Measurement of the voltage drop enables the current flowing in the circuit to be monitored, provided the resistance of the current sense resistor is known. Once the current flowing through the load is known, the measurement of a second voltage drop across the load may be performed. By multiplying the series current with the second voltage drop, the power dissipation in the load may be calculated.

Conventionally, the voltage drop across the current-sense resistor is rather small. Accurate monitoring circuitry is, thus, required to monitor the voltage drop. Typically, a sensitive instrumentation amplifier including several operational amplifiers and their associated passive components, are required to amplify the voltage drop. To convert an analogue version of the voltage drop into a digital signal useful for computation purposes, the output of the instrumentation amplifier is connected to an analogue-to-digital converter (ADC). Thus, a current flowing in series into a load may be measured, but at a significant component cost. The series measuring resistor causes an additional parasitic power loss, which makes the power measurement inaccurate.

Systems employing high-efficiency loads (such as piezo-electric elements) often require a high driving voltage. Thus, a step-up regulator (voltage converter) is used to provide a voltage at a suitable level for driving the high-efficiency load. The voltage converter feeds an amplifier to drive the high-efficiency load (piezo-electric element). A microcontroller can be used to generate pulse-width-modulated (PWM) signals, for driving such a DC step-up regulator. This microcontroller can also be used to calculate an indication of the power dissipation in a high-efficiency load (piezo-electric element), by analyzing the PWM signal.

Towards this end, a first aspect of the invention provides a voltage converter control and power dissipation monitoring arrangement for monitoring an efficient load comprising: a voltage converter 10 and a processor 12.

The processor 12 is configured to obtain, via a first input 16 of the processor, an input voltage level of a voltage applied to an input 18 of the voltage converter 10. The processor 12 is configured to generate a voltage converter control signal 24 for controlling the level of an output voltage 20 of the voltage converter 10, wherein the output 20 of the voltage converter is configured to provide an output voltage, being a multiple of the input voltage level, to an external load terminal 22 of the voltage converter 10, based on a voltage level defined by a voltage converter control signal 24. The voltage converter control signal 24 is input into a voltage control input of the voltage converter 10, and the voltage converter control and power dissipation monitoring arrangement is configured to generate an averaged voltage signal of the voltage converter control signal 24.

The processor 12 is configured to generate a load dissipation metric indicative of power dissipated in load circuitry connected to the external load terminal 22, by multiplying the input voltage level with the smoothed voltage signal.

Thus, the power dissipated in a high-efficiency load (piezo-electric element) is measured indirectly. This indirect measurement is accurate, because the circuitry associated with the drive unit of a high-efficiency load (a piezo-electric drive unit, such as a class E amplifier) is typically very efficient. Therefore, the power dissipated in the high-efficiency load is almost the same as that predicted by the voltage feeding the load.

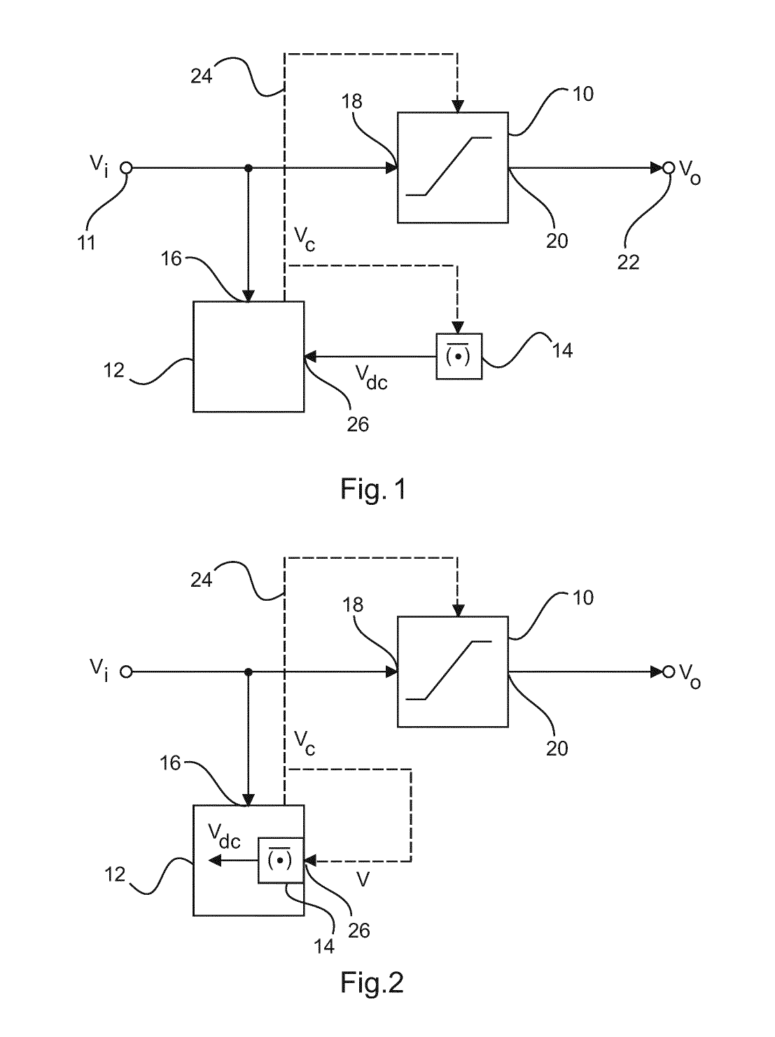

FIG. 1 shows a voltage converter control and power dissipation monitoring arrangement according to a first aspect of the invention. An input voltage, Vᵢ, is input at an input terminal 11. The input voltage is applied to the input terminal 18 of a voltage converter 10. An output 20 of the voltage converter 10 is applied to an external load terminal 22, across which an output voltage Vₒ appears.

The voltage converter 10 functions to step-up the input voltage of Vᵢ. Typical topologies of circuits used to achieve this are buck-boost converters (step-up converters). The voltage converter 10 is controlled by a voltage converter control signal denoted by a dotted line 24. The voltage converter control signal is a pulse width modulated (PWM) signal generated by the processor 12. When the voltage converter control signal has a low mark-space ratio, the output voltage of the voltage converter 10 will be relatively low. When the mark-space ratio of the voltage converter 10 is relatively high, the voltage output Vₒ will be relatively high. Therefore, by using the processor 12 to control the mark-space ratio of the voltage converter control signal, it is possible to vary the output voltage of the voltage converter. For a typical step-up converter, there is a substantially linear relationship between the mark-space ratio of the pulse width modulation of the voltage converter control signal, and the output voltage output from the voltage converter 10.

Also shown in FIG. 1 is a processor 12. The processor may be a microcontroller, a microprocessor, or may be implemented in a field programmable gate array (FPGA) or using CPLD logic. The processor 12 in FIG. 1 obtains an input voltage level of a voltage applied to an input 18 with a voltage converter at the input 16 of the processor. It will be appreciated that the voltage Vᵢ will be an analogue value. Therefore, the processor may use an analogue to digital converter (ADC) input pin of the processor 12 to convert this signal into a digital value before use inside the processor. Alternatively, an external ADC may be used to sample the input 18 of the voltage converter, and a digital value provided either in serial or parallel format may be applied at input 16 of the processor.

It will be appreciated that the processor 12 may perform load power monitoring functions and voltage converter control operations simultaneously. As is known to the person skilled in the art, this plurality of operations may be implemented in multiple threads or memory spaces of the processor, or by using a real-time operating system. In other words, the same processor may be used to monitor the load dissipation metric, and to control the voltage converter 10.

A signal smoothing function of the voltage converter control and power dissipation monitoring arrangement may, in one embodiment, be an analogue filter, such as a RC network. In such a network, the pulse width modulated signal at 24 would be smoothed to provide a substantially stable signal which would be a DC level, increasing as the mark-space ratio increases, and decreasing as the mark-space ratio decreases. The design of such RC networks relies on a selection of components suited to the operating frequency of the PWM voltage converter control signal, as is known to the person skilled in the art.

In the case of an analogue signal smoother, the processor 12 will require another ADC input (separate from the processor input 16) at input 26 to convert the analogue smoothed DC signal into a digital signal capable of being used in the internal arithmetic circuits of the processor. Microprocessors with a plurality of ADC inputs are known. Alternatively, an external ADC could be provided at input 26 to perform the conversion, and the digital value of the smoothed DC signal would be input into the processor 12.

Furthermore, an embodiment comprising an analogue multiplexer is provided, wherein the input voltage and the smoothed DC signal are connected to inputs of the analogue multiplexer. The analogue multiplexer would switch between the input voltage and the smoothed DC voltage. Provided the switching was timed to occur during a steady state of circuit operation (so that the dissipation by the high-efficiency load was the same), this would enable the input voltage and smoothed DC voltage to be sampled by the same ADC or ADC input pin.

In operation, the circuit shown in FIG. 1 will have the voltage Vᵢ applied across input terminal 11. A current will flow into the input terminal 18 of the voltage converter 10, and the voltage converter (step-up regulator) will function to boost the voltage which will be output at terminal 22 as Vₒ. Adjustments to the output voltage are made by causing the processor 12 to vary the mark-space ratio of the pulse width modulated voltage converter control signal 24. The demand for such a voltage adjustment may come from application microcode executing on a microprocessor, for example. The signal smoother 14 converts the voltage converter control signal into a DC average value, whose level varies dependent on the mark-space ratio of the voltage converter control signal 24. The signal smoother outputs the smoothed voltage signal of the voltage converter control signal V_{DC}, which is then input into input 26 of the processor 12.

Therefore, it is possible for the processor 12 to acquire one, or a set of synchronized samples of the input voltage Vᵢ, and the smoothed voltage signal V_{dc}.

An indication of the power dissipation in a load connected to the load terminal 22 is proportional to the average DC level multiplied at processor input 26 multiplied by the input voltage at the processor input 16.

This relation gives the microcontroller a way of tracking an indication of the output power of the circuit, irrespective of the input voltage.

The voltage converter control signal 24 generated by the processor 12 is typically a fixed frequency, with a varying mark-space ratio. For a fixed input voltage Vᵢ, the pulse width modulated signal to a step-up regulator is proportional to the power emerging from the step-up regulator. As the mark-space ratio of the voltage converter control signal 24 increases, the output voltage will increase accordingly.

According to an embodiment, the processor 12 takes a time-windowed average of the input voltage Vᵢ and the DC-averaged voltage converter control signal 24. This can improve the stability of the estimated load dissipation metric.

At a fixed output power, the PWM signal to the voltage converter 10 is proportional to the converter's supply voltage. The lower the supply voltage, the higher the pulse width modulated signal will be, in order to maintain a fixed output voltage from the voltage converter 10. The following examples show how this simple equation can be used by a microcontroller to measure the power delivered by a step-up regulator, with no influence from Vᵢ.

Therefore, by measuring the voltage converter control signal 24, and the input voltage of the voltage converter 10, an indication of the load power can be measured indirectly. When the load is assumed to be efficient, this provides an accurate estimate of power dissipated in the load.

According to an embodiment of the invention, a voltage converter control and power dissipation monitoring arrangement for monitoring an efficient load is provided as discussed above, wherein the monitoring arrangement is configured to be connected to a nebulizer element, a nebulizer transducer, a piezo-electric element, or a piezo-electric loudspeaker.

FIG. 2 shows an alternative circuit configuration having a substantially equivalent functionality for a voltage converter control and power dissipation monitoring arrangement described previously. FIG. 2 shows a voltage converter control and power dissipation monitoring arrangement comprising a voltage converter 10, and a processor 12. In this case, the signal smoothing function 14 is internal to the processor 12. In one embodiment, the signal smoother 14 samples the voltage converter control signal 24 generated by the processor 12 on a digital pin input of the processor 12. The digital pin input provides the pulse width modulated signal 24 to arithmetic calculation circuitry in the processor 12, which can be performed using the usual registers of the processor as known by the person skilled in the art. Thus, the signal smoother function 14 in this alternative arrangement is effectively a DC smoothing filter implemented in digital arithmetic. Of course, the voltage converter control signal 24 may be provided to the signal smoother 14 internally in the processor 12, by means of a processor register variable, for example. Alternatively, if another means is used to generate the voltage converter control signal, this may be input via a digital input pin of the processor 12.

Thus, a voltage converter control and power dissipation monitoring arrangement according to the previous description has been presented, wherein the signal smoother 14 is provided as a digital averaging circuit implemented using the processor 10.

According to an embodiment of the invention, voltage converter control and power dissipation monitoring arrangement as described previously is provided, wherein the signal smoother 14 is provided as a digital arithmetic arrangement inside the processor 12, and wherein the voltage control signal generated by the processor 10 is input into a third input of the processor and provided to the digital arithmetic arrangement.

The previously discussed aspect, and embodiments, may also be considered as a voltage converter control and power dissipation monitoring arrangement for monitoring a nebulizer element, or a piezo-electric element.

According to an aspect of the invention, there is provided a nebulizer system 40, comprising a nebulizer element 42, a nebulizer controller 44, and a voltage converter control and power dissipation monitoring arrangement for monitoring an efficient load, as discussed in the previous first aspect, and its embodiments.

A power supply of the nebulizer element is connected to the external load terminal of the voltage converter 10 comprised in the voltage converter control and power dissipation monitoring arrangement, and wherein the load dissipation metric calculated by the arrangement is used by the nebulizer controller to control a nebulization signal applied to the nebulizer element.

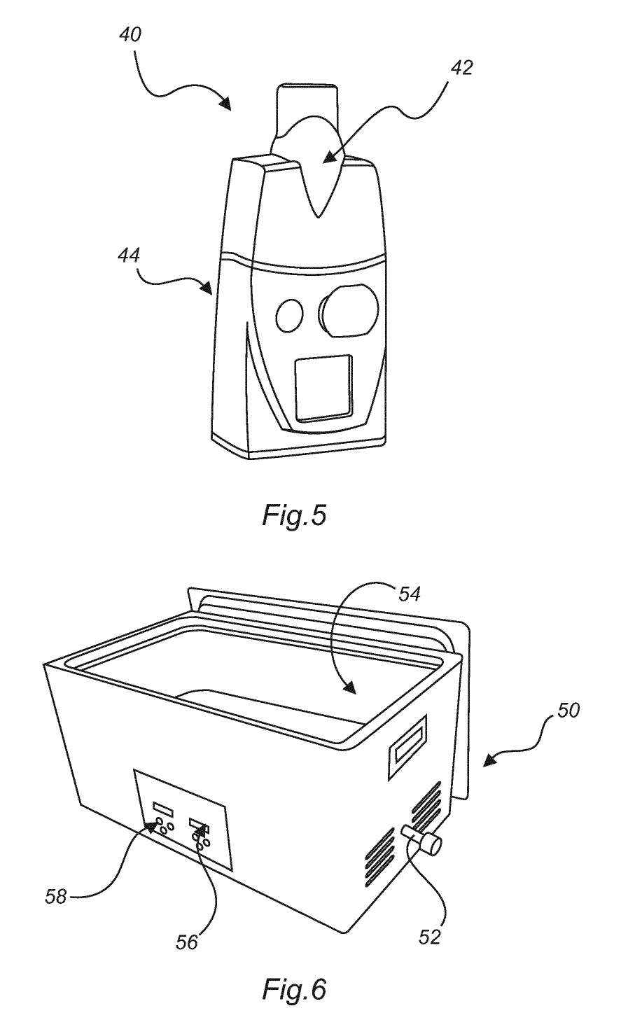

FIG. 5 shows a nebulizer system 40 according to a second aspect of the invention. The nebulizer system 40 comprises a nebulizer element 42, a nebulizer controller 44, and a voltage converter control and power dissipation monitoring arrangement as previously described (not shown in FIG. 5).

A power supply of the nebulizer element is connected to the external load terminal of the voltage converter 10 comprised in the voltage converter control and power dissipation monitoring arrangement, and the load dissipation metric calculated by the voltage converter control and power dissipation monitoring arrangement is used by the nebulizer controller to control a nebulization signal applied to the nebulizer element.

In an example of a nebulizer system, a nebulizer element comprising a piezo-electric element is housed in a vessel in fluid communication with a liquid medicament supply 44. The mouthpiece 42 dispenses an aerosol entrained medicament to a patient.

According to an embodiment of the invention, the nebulizer system 40 is provided as described previously, and the voltage converter 10 is configured to provide, via the external load terminal, a nebulizer load drive signal to the nebulizer element 42.

According to an embodiment of the invention, a nebulizer system as described previously is provided, wherein the nebulizer element 30 comprises an amplifier 32 electrically connected to a nebulizer transducer 34. The output 20 of the voltage converter 10 is configured to provide a supply voltage to the amplifier, and an output of the amplifier 32 is configured to provide a drive signal to the nebulizer transducer 34, wherein the load signal is generated from an amplifier input signal Vₛ.

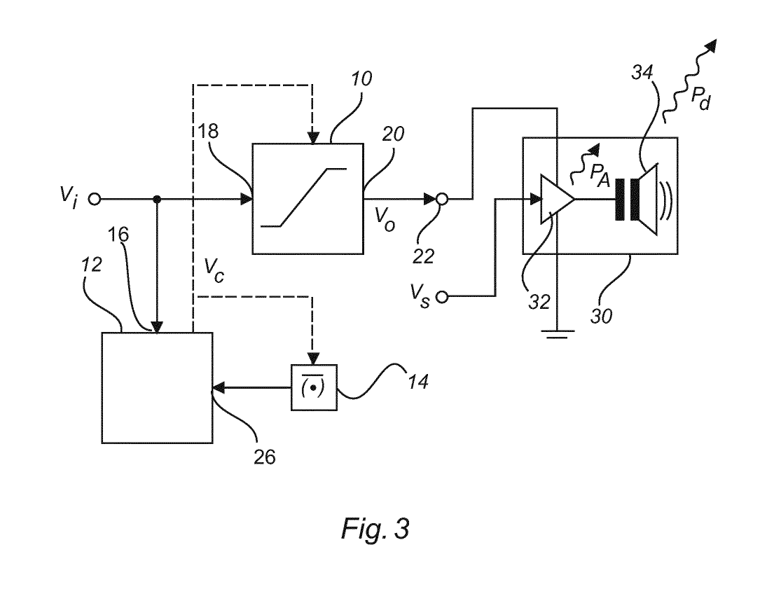

FIG. 3 shows an embodiment of a circuit used in a nebulizer system. This is a voltage converter control and power dissipation monitoring arrangement for monitoring an efficient load for use in the nebulizer system of the second aspect. The components of the voltage converter control and power dissipation monitoring arrangement for monitoring an efficient load shown in FIG. 1 apply also to the system of FIG. 3.

Thus, the addition to the embodiment of FIG. 1 is a nebulizer element 30, which comprises an amplifier 32 connected to a nebulizer transducer 34. The supply voltage Vₒ is applied to the supply rail of the amplifier 32, and the other supply rail is grounded (or alternatively connected to a negative voltage).

The signal Vₛ is provided from an external means to provide a suitable signal to drive the load 30 (high-efficiency transducer), such as a digital signal processor, or a microprocessor. For example, in the nebulizer system, Vₛ may be a signal suitable for providing dispersion of a liquid medicament into an aerosol medicament.

According to an embodiment of the invention, the amplifier 32 has efficiency above 80%.

Typically, a class E amplifier may be used to power the transducer 34. Class E amplifiers have high power efficiency. If an amplifier having high power efficiency is used, the dissipation Pₐ from the amplifier will be low, compared to the dissipation P_{d} from the transducer 34. In effect, the voltage converter control and power dissipation monitoring arrangement is able to deduce the power dissipation in the high efficiency transducer 34 more accurately, because so little power is dissipated in other components (such as the amplifier 32) in the nebulizer element.

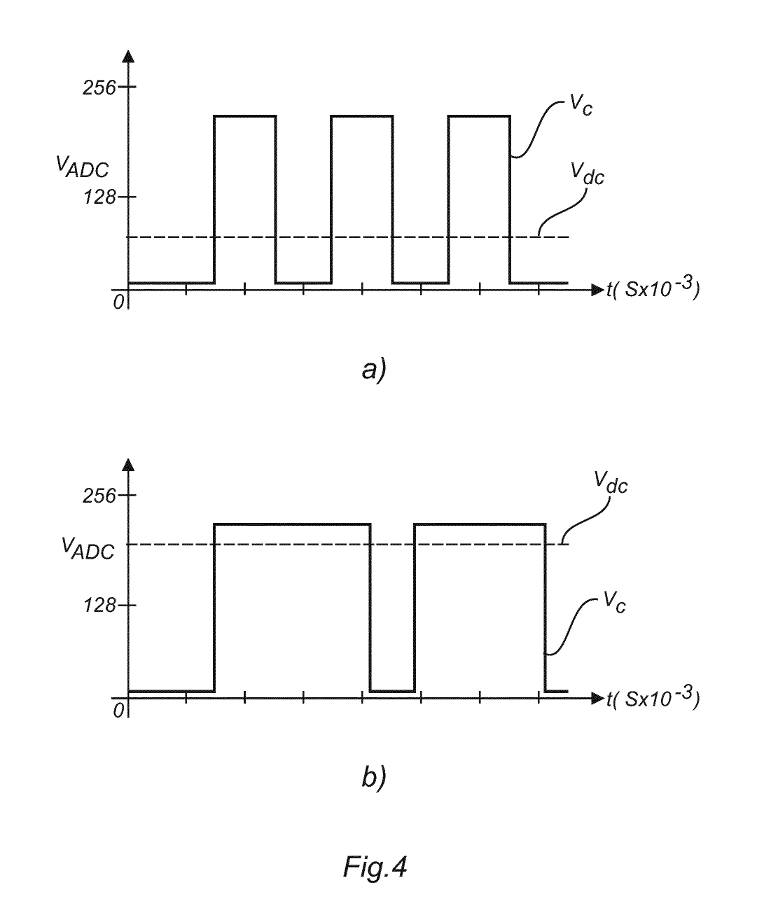

FIG. 4 shows two plots providing an example of the operation of a voltage converter control and power dissipation monitoring arrangement for monitoring an efficient load.

The solid line represents the voltage converter control signal V_{c} 24 which drives the voltage converter. The dotted line V_{dc} represents the smoothed voltage signal provided by the signal smoother 14. In FIG. 4A, the mark-space ratio is relatively low, because the regions where the signal V_{c} is low are longer in duration than the regions where the signal V_{c} is high. Accordingly, the smoothed voltage signal will be relatively low. (The units on the x-axis V are in bits of an 8-bit ADC).

Turning to FIG. 4B, the mark-space ratio here is relatively high, because the areas where the voltage control input is high are greater in duration than the regions where the voltage control input is low. Therefore, the signal V_{dc} in FIG. 4B is much higher. It is noted that this result will occur using correctly designed analogue filtering circuits, such as RC smoothing circuits, or from the application of an appropriate digital averaging algorithm.

According to an embodiment of the invention, a nebulizer system 40 as described previously is provided wherein the amplifier has efficiency above 80%.

According to an embodiment of the invention, a nebulizer system 40 as described previously is provided wherein the high-efficiency transducer 34 is a piezo-electric element.

Thus, the high-efficiency transducer dissipates power mostly in the form of useful work, rather than in the form of parasitic losses.

According to this aspect of the invention, a nebulizer system is provided which is able effectively to calculate nebulizer parameters (such as an estimation of the total medicament delivered to a patient) using a load dissipation metric calculated in the manner described. The load dissipation metric can be calculated without the use of a current-sense resistor and instrumentation electronics. Instead, the load dissipation metric may be calculated using existing power supply circuitry used to control the nebulizer system.

According to an embodiment of the invention, the load dissipation metric is used to monitor the performance of a nebulizer element.

According to an embodiment of the invention, the load dissipation metric is used to monitor the performance of a nebulizer.

According to another aspect of the invention, there is provided a piezo-electric speaker system. The system comprises a piezo-electric loudspeaker element, a loudspeaker controller, and a voltage converter control and power dissipation monitoring arrangement as discussed previously.

The external load terminal of the voltage converter comprised in the voltage converter control and power dissipation monitoring arrangement is connected to the piezo-electric loudspeaker element.

The load dissipation metric calculated by the voltage converter control and power dissipation monitoring arrangement is used by the loudspeaker controller to control the amplitude of an audio signal applied to the piezo-electric loudspeaker element.

According to this aspect of the invention, a piezo-electric speaker system may be provided with an output power control (volume control) without using current-sense resistor techniques. A piezo-electric loudspeaker is very efficient when emitting sound, and so it can be assumed that most of the power dissipated in the load results from the emission of sound waves. Therefore, the volume of a piezo-electric speaker system can be monitored without complicated electronics.

In a piezo-electric loudspeaker system, the signal Vₛ shown in FIG. 3 may be an audio signal for broadcast, for example.

According to an aspect of the invention, there is provided an ultrasonic cleaning system 50. The ultrasonic cleaning system comprises an ultrasonic cleaning element 52 housed in an ultrasonic cleaning bath 54. The ultrasonic cleaning system further comprises an ultrasonic cleaning controller 56 and the voltage converter control and power dissipation monitoring arrangement 58 as previously described.

The external load terminal of the voltage converter comprised in the voltage converter control and power dissipation monitoring arrangement is connected to an ultrasonic cleaning element. The load dissipation metric calculated by the voltage converter control and power dissipation monitoring arrangement is used by the ultrasonic cleaning controller to control the amplitude of a cleaning signal applied to an ultrasonic cleaning element.

Accurate control of the ultrasonic cleaning power is important in an ultrasonic cleaning system, because certain items may be damaged by the application of an ultrasonic cleaning field of too high a magnitude. Because the ultrasonic cleaning element in an ultrasonic cleaning system is typically a high-efficiency transducer such as a piezo-electric sounder, the application of a voltage converter control and power dissipation monitoring arrangement for monitoring an efficient load as discussed above enables the accurate estimation of the power applied to the ultrasonic cleaning bath.

In an ultrasonic cleaning system, the signal Vₛ shown in FIG. 3 may be an ultrasonic signal optimized for cleaning components in an ultrasonic water bath, for example.

According to a fifth aspect of the invention, a method for voltage converter control and power dissipation monitoring for monitoring an efficient load. The method comprises steps of:

a) obtaining (60) an input voltage level of a voltage applied to an input of a voltage converter;
b) generating (62) a voltage converter control signal for controlling the level of an output voltage;
c) providing (64) an output voltage, being a multiple of the input voltage level, to an external load terminal based on a voltage level defined by the voltage converter control signal;
d) generating (66) an averaged voltage signal of the voltage converter control signal;
e) generating (68) a load dissipation metric indicative of power dissipated in an external load connected to the external load terminal by multiplying the input voltage level with the averaged voltage signal.

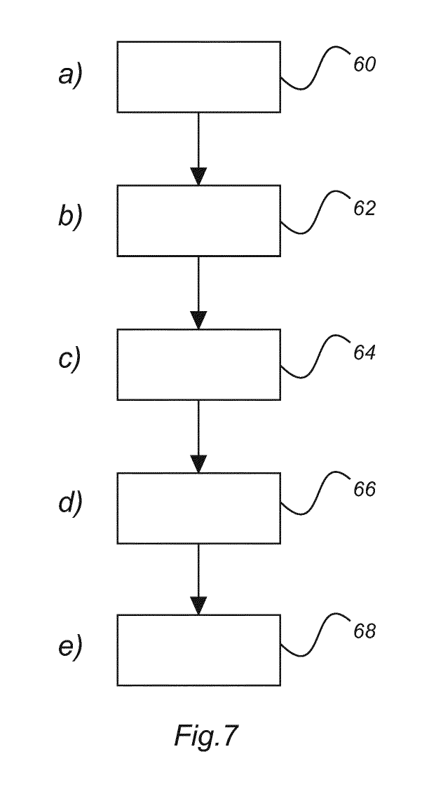

FIG. 7 illustrates the method according to the fifth aspect of the invention.

According to an embodiment of the invention, a method is provided as discussed above, wherein the load element is connected to the external load terminal; and wherein the voltage converter provides, via the external load terminal, the output voltage to the load element.

According to an embodiment, a method is provided as discussed above, wherein the load element 30 comprises an amplifier 32 and a transducer 34, wherein the output 20 of the voltage converter 10 is provides a supply voltage to the amplifier, and an output of the amplifier provides a drive signal to the transducer 34, wherein the load signal is generated from an amplifier input signal.

According to an embodiment, a method is provided as discussed above, wherein the amplifier 32 has an efficiency above 80%.

According to an embodiment, a method is provided as discussed above, wherein the transducer 34 is a piezo-electric element.

According to an embodiment, a method is provided as discussed above, wherein a signal smoother 14 is provided as a filter circuit connected between the voltage control signal input 24 and the second input 26 of the processor 12.

According to an embodiment, a method is provided as discussed above, wherein a signal smoother 14 is provided as a digital arithmetic arrangement inside the processor 12, and wherein the voltage control signal is input into a third input of the processor and provided to the digital arithmetic arrangement.

According to an embodiment, a method is provided as described previously, further comprising after step d) the steps of:

d1) providing a supply voltage to a supply input of an amplifier; and
d2) providing a load signal for driving a transducer from the output of the amplifier, wherein the load signal is generated from an amplifier input signal.

According to an embodiment of the invention, the transducer of step d2) may be a nebulizer element, a piezo-electric loudspeaker, a piezo-electric transducer, or a piezo-electric load element for use in an ultrasonic bath.

According to an aspect of the invention, a computer program element is provided for controlling a voltage converter control and power dissipation monitoring arrangement according to the previous description, which, when being executed by a processing unit, is adapted to perform the method steps as previously described.

According to an aspect of the invention, a computer-readable medium having stored the program of the previous description is provided.

A computer program element might be stored on a computer unit which could also be an embodiment of the invention. The computing unit may be adapted to perform or induce performance of the steps of the method described above. Moreover, it may be adapted to operate the components of the above-described apparatus.

The computing unit can be adapted to operate automatically and/or to execute the orders of a user. A computer program may be loaded into a working memory or a data processor. The data processor may thus be equipped to carry out the method of the invention.

The computing unit can be supplemented with a high performance processing unit such as a graphics card, or an FPGA extension card, to perform computationally intensive operations. This exemplary embodiment of the invention covers both the computer program that has the invention installed from the beginning, and a computer program that by means of an update turns an existing program into a program that uses the invention.

A computer program may be stored and/or distributed on a suitable medium, such as an optical storage media, or a solid state medium supplied together with, or as a part of other hardware, but may also be distributed in other forms, such as via the Internet or other wired or wireless telecommunication systems.

The computer program may also be presented over a network like the World Wide Web, and can be downloaded into the working memory of a data processor from such a network.

According to a further exemplary embodiment of the present invention, a medium for making a computer program element available for downloading is provided, which computer program element is arranged to perform a method according to one of the previously described embodiments of the invention.

### EXAMPLES

The following examples deal with four cases of a voltage converter driving a load via a class E amplifier.

According to a first example, the case of a step-up amplifier (feeding an nebulizer) having a supply voltage of 20V, and drawing a current of 100 mA is considered. In this case, the power dissipated in the nebulizer will be P=VI=2 W.

In this example, the battery supply of the voltage converter is 3 V. Thus, the power to supply the nebulizer amplifier, assuming an 80% efficiency, is 2 W*1.2=2.4 W.

If the DC average of the PWM signal needed to deliver 2.4 W at a voltage converter supply voltage of 3.0 V is 200 (PWM level=200 A/D samples), the processor multiplies 200*3.0 to yield 600 A/D samples.

Thus, in these conditions, an nebulizer output power of 2 W has a processor calculated number (load dissipation metric) of 600.

According to a second example, the case of a step-up amplifier (feeding an nebulizer) having a supply voltage of 20V, and drawing a current of 100 mA is considered. In this case, the power dissipated in the nebulizer will be P=VI=2 W.

In this example, the battery supply of the voltage converter is 2.5 V. Thus, the power to supply the nebulizer amplifier, assuming an 80% efficiency, is 2 W*1.2=2.4 W.

If the DC average of the PWM signal needed to deliver 2.4 W at a voltage converter supply voltage of 2.5 V is 200 A/D samples*(3.0V/2.5V)=240 (PWM level), the processor calculates 240*2.5 to yield 600 A/D samples.

Thus, in these conditions, an nebulizer output power of 2 W has a processor calculated number (load dissipation metric) of 600.

Therefore, a change in the battery supply input voltage does not change the nebulizer power calculation, as expressed in the load dissipation metric.

According to a third example, the case of a step-up amplifier (feeding an nebulizer) having a supply voltage of 20V, and drawing a current of 150 mA is considered. In this case, the power dissipated in the nebulizer will be P=VI=3 W.

In this example, the battery supply of the voltage converter is 3.0 V. Thus, the power to supply the nebulizer amplifier, assuming an 80% efficiency, is 2 W*1.2=3.6 W.

For the same DC input voltage of 3.0V, the DC average of the PWM signal needed to deliver 3.6 W will now be (3.6/2.4) W—1.5 times higher than in the foregoing examples. So, 200 PWM*1.5 gives a PWM value of 300. The processor multiplies the 200 by the battery supply input to yield 300*3.0V=900.

In these conditions, a nebulizer output power of 3 W has a processor calculated load dissipation metric of 900.

According to a fourth example, the case of a step-up amplifier (feeding an nebulizer) having a supply voltage of 20V, and drawing a current of 150 mA is considered. In this case, the power dissipated in the nebulizer will be P=VI=3 W.

In a case where the battery supply is 2.5V, the power out to supply the nebulizer amplifier is 3 W*1.2 (assuming an 80% amplifier efficiency)=3.6 W.

The DC average (or the PWM signal) needed to deliver 3.6 W will now be (3.0V/2.5V)—1.2 times higher than the above case in example 3. Therefore, a PWM value of 300*1.2 provides a PWM value of 360. The processor multiplies the PWM value of 360 by the battery input of 2.5V to yield a load dissipation metric of 900. Thus, for these conditions, an nebulizer output power of 3 W has a processor calculated value of 900. This does not change with the battery input—rather, it gives a value for nebulizer power.

It should to be noted that embodiments of the invention are described with reference to different subject-matter. In particular, some embodiments are described with reference to method-type claims, whereas other embodiments are described with reference to the device-type claims.

A person skilled in the art will gather from the above, and the following description that, unless otherwise notified, in addition to any combination of features belonging to one type of subject-matter, also any other combination between features relating to different subject-matters is considered to be disclosed with this application.

All features can be combined to provide a synergetic effect that is more than the simple summation of the features. While the invention has been illustrated and described in detail in the drawings and foregoing description, such illustrations and descriptions are to be considered illustrative, or exemplary, and not restrictive. The invention is not limited to the disclosed embodiments. Other variations to the disclosed embodiments can be understood, and effected by those skilled in the art in practicing the invention, from a study of the drawings, the disclosure, and the dependent claims.

In the claims, the word “comprising” does not exclude other elements or steps, and the indefinite article “a” or “an” does not exclude a plurality. A single processor, or other unit, may fulfil the functions of several items recited in the claims. The mere fact that certain measures are recited in mutually different dependent claims does not indicate that a combination of these measures cannot be used to advantage. Any reference signs in the claims should not be construed as limiting the scope.

1. A voltage converter control and power dissipation monitoring arrangement for monitoring an efficient load comprising:
a voltage converter; and
a processor;
wherein the processor is configured to obtain, via a first input of the processor, an input voltage level of a voltage applied to an input of the voltage converter;
wherein the processor is configured to generate a PWM voltage converter control signal for controlling the level of an output voltage of the voltage converter;
wherein the output of the voltage converter is configured to provide an output voltage, being a multiple of the input voltage level, to an external load terminal of the voltage converter, based on a voltage level defined by the PWM voltage converter control signal, wherein the PWM voltage converter control signal is input into a voltage control input of the voltage converter;
wherein the PWM voltage converter control and power dissipation monitoring arrangement is configured to generate an averaged voltage signal of the PWM voltage converter control signal;
wherein the processor is configured to generate a load dissipation metric indicative of power dissipated in a load element connected to the external load terminal, by multiplying the input voltage level with the averaged voltage signal.

2. The voltage converter control and power dissipation monitoring arrangement of claim 1, wherein the averaged voltage signal is provided by a signal smoother, and wherein the signal smoother is provided as a filter circuit connected between the voltage control signal input and a second input of the processor.

3. The voltage converter control and power dissipation monitoring arrangement of claim 1, wherein the averaged voltage signal is provided by a signal smoother; wherein the signal smoother is provided as a digital arithmetic arrangement inside the processor; and wherein the voltage control signal is input into a third input of the processor and provided to the digital arithmetic arrangement of the processor.

4. A nebulizer system, comprising:
a nebulizer element;
a nebulizer controller;
a voltage converter control and power dissipation monitoring arrangement according to any preceding claims; wherein a power supply of the nebulizer element is connected to the external load terminal of the voltage converter comprised in the voltage converter control and power dissipation monitoring arrangement; and wherein the load dissipation metric calculated by the voltage converter control and power dissipation monitoring arrangement is used by the nebulizer controller to control a nebulization signal applied to the nebulizer element.

5. The nebulizer system of claim 4, wherein the voltage converter is configured to provide, via the external load terminal, a nebulizer load drive signal to the nebulizer element.

6. The nebulizer system of claim 4, wherein the nebulizer element further comprises:
an amplifier; and
a nebulizer transducer;
wherein the output of the voltage converter is configured to provide a supply voltage to the amplifier, and an output of the amplifier is configured to provide a drive signal to the nebulizer transducer, wherein the load signal is generated from an amplifier input signal.

7. The nebulizer system of claim 4, wherein the amplifier has an efficiency of above 80%.

8. The nebulizer system of claim 4, wherein the nebulizer transducer is a piezo-electric element.

9. A piezo-electric speaker system, comprising:
a piezo-electric loudspeaker element;
a loudspeaker controller;
a voltage converter control and power dissipation monitoring arrangement for monitoring an efficient load according to claim 1;
wherein the external load terminal of the voltage converter comprised in the voltage converter control and power dissipation monitoring arrangement for monitoring an efficient load is connected to the piezo-electric loudspeaker element;
wherein the load dissipation metric calculated by the voltage converter control and power dissipation monitoring arrangement for monitoring an efficient load is used by the loudspeaker controller to control the amplitude of an audio signal applied to the piezo-electric loudspeaker element.

10. An ultrasonic cleaning system, comprising
an ultrasonic cleaning element housed in an ultrasonic cleaning bath;
an ultrasonic cleaning controller;
a voltage converter control and power dissipation monitoring arrangement for monitoring an efficient load according to claim 1;
wherein the external load terminal of the voltage converter comprised in the voltage converter control and power dissipation monitoring arrangement for monitoring an efficient load is connected to an ultrasonic cleaning element; wherein the load dissipation metric calculated by the voltage converter control and power dissipation monitoring arrangement for monitoring an efficient load is used by the ultrasonic cleaning controller to control the amplitude of a cleaning signal applied to the ultrasonic cleaning element.

11. A method for voltage converter control and power dissipation monitoring for monitoring an efficient load comprising the steps of:
a) obtaining an input voltage level of a voltage applied to an input of a voltage converter;
b) generating a PWM voltage converter control signal for controlling the level of an output voltage;
c) providing an output voltage, being a multiple of the input voltage level, to an external load terminal based on a voltage level defined by the PWM voltage converter control signal;
d) generating an averaged voltage signal of the PWM voltage converter control signal;
e) generating a load dissipation metric indicative of power dissipated in an external load connected to the external load terminal by multiplying the input voltage level with the averaged voltage signal.

12. The method of claim 11, further comprising the step of: f) providing electrical power to a load element using the voltage converter based on a voltage level defined by the PWM voltage converter control signal.

13. The method of claim 12, further comprising after step d) the steps of:
d1) providing a supply voltage to an supply input of an amplifier; and
d2) providing a load signal for driving a transducer from the output of the amplifier, wherein the load signal is generated from an amplifier input signal.

14. (canceled)

15. A computer readable medium having stored the computer program element of claim 14.
